SEQUENCE LISTING (4 sequences)

SEQ ID NO: 1 - protein, 170 aa, Homo sapiens
MLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPEDFPDDNQNSKKLAAGHELQPLAIVDQRPSSRASSRASSRPRPDDLEI

SEQ ID NO: 2 - protein, 10 aa, synthetic construct
DDDDDDDDDD

SEQ ID NO: 3 - protein, 180 aa, synthetic construct
MHHHHHHGGGGSGGGGSVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEIGGSGGSDDDDDDDDDD

SEQ ID NO: 4 - DNA, 534 bp, synthetic construct
  source 1..534: mol_type = other DNA
atgcatcatcaccatcaccacatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctctatctcctcctgggtacaagctggttactggcgacagaaacaattcttcttgccgcaattacaacaagcaagcaagtgagcaaaactgggctaattacagtgcagaacaaaatcgactagggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctag

(19) United States
(12) Patent Application Publication
(10) Pub. No.: US 2025/0289864 A1
(43) Pub. Date: Sep. 18, 2025

(54) GJA1-20K TO LIMITED CARDIAC ARRHYTHMIAS

(71) Applicant: UNIVERSITY OF UTAH RESEARCH FOUNDATION, Salt Lake City, UT (US)

(72) Inventors: Robin M. Shaw, Salt Lake City, UT (US); Joseph A. Palatinus, Salt Lake City, UT (US); Guillaume Hoareau, Salt Lake City, UT (US)

(21) Appl. No.: 18/861,504

(22) PCT Filed: Nov. 10, 2022
(86) PCT No.: PCT/US2022/079667

§ 371 Date: Oct. 29, 2024

Related U.S. Application Data
(60) Provisional application No. 63/341,551, filed on May 13, 2022.

(51) Int. Cl. C07K 14/705; A61K 38/00; A61P 9/06

(52) U.S. Cl. CPC C07K 14/705; A61P 9/06; A61K 38/00

(57) ABSTRACT

Described herein are compositions and methods for treating, preventing, reducing the likelihood of having, reducing the severity of and/or slowing the progression of a medical condition related to cardiac arrhythmia syndromes in a subject using GJA1-20k as a therapeutic agent. In one embodiment, the compositions and methods comprise a GJA1-20k peptide therapy. In another embodiment, the compositions and methods comprise a GJA1-20k gene therapy.

### CROSS-REFERENCE TO RELATED APPLICATIONS

This application claims priority to U.S. Provisional Patent Application No. 63/341,551, filed on May 13, 2022, the entire contents of which are fully incorporated herein by reference.

### FEDERALLY SPONSORED RESEARCH

This invention was made with government support under grants HL138577 and HL155886 awarded by the National Institutes of Health. The government has certain rights in this invention.

### REFERENCE TO SEQUENCE LISTING

This application was filed with a Sequence Listing XML in ST.26 XML format in accordance with 37 C.F.R. § 1.821. The Sequence Listing XML file submitted in the USPTO Patent Center, “026389-9329-WO01_sequence_listing_XML_26-OCT-2022.xml,” was created on Oct. 26, 2022, contains 4 sequences, has a file size of 6.0 Kbytes, and is hereby incorporated by reference in its entirety into the specification.

### TECHNICAL FIELD

Described herein are compositions and methods for treating, preventing, reducing the likelihood of having, reducing the severity of and/or slowing the progression of a medical condition related to cardiac arrhythmia syndromes in a subject using GJA1-20k as a therapeutic agent. In one embodiment, the compositions and methods comprise a GJA1-20k peptide therapy. In another embodiment, the compositions and methods comprise a GJA1-20k gene therapy.

### BACKGROUND

Existing pharmaceutical therapies are inadequate for treating many cardiac arrhythmia syndromes, including ventricular tachycardia in failing hearts and hearts with arrhythmogenic cardiomyopathy (ACM), which is an under-recognized inherited disorder of desmosomes that presents in early adulthood. Current therapies for treating these arrhythmias include oral and intravenous administration of pharmaceutical drugs such as amiodarone, lidocaine, or sotolol.

Most arrhythmia syndromes trace their cellular origins to a lack of cell-cell Connexin 43 (Cx43) gap junction coupling, as precise Cx43 gap junction formation and localization seem to be critical for proper intercellular communication and electrophysiological functioning in the heart. It has previously been reported that N-terminally truncated Cx43 isoforms exist endogenously in the heart, and that they are produced by alternative translation of the GJA1 mRNA. The most abundant internally translated Cx43 isoform, named GJA1-20k, is an auxiliary subunit for the trafficking of Cx43 in heterologous expression systems, and GJA1-20k was previously found to be required for Cx43 trafficking and gap junction formation.

Thus, what is needed are new GJA1-20k compositions and methods for treating acute and chronic cardiac arrhythmias in patients with acquired or genetic predisposition. Such compositions and methods would be useful in treating a variety of cardiac arrhythmia syndromes.

### SUMMARY

One embodiment described herein is a method of treating, preventing, reducing the likelihood of having, reducing the severity of, and/or slowing the progression of a cardiac arrhythmia syndrome in a subject, the method comprising: administering to the subject a therapeutically effective amount of a pharmaceutical composition comprising: a GJA1-20k polypeptide or functional variant or fragment thereof; or a GJA1-20k gene expression vector comprising a polynucleotide sequence encoding a GJA1-20k polypeptide or functional variant or fragment thereof. In one aspect, the pharmaceutical composition is administered to the subject by intravenous (IV) injection. In another aspect, the GJA1-20k polypeptide or functional variant or fragment thereof comprises an amino acid sequence having at least 90-99% identity to any one of SEQ ID NO: 1 or 3. In another aspect, the GJA1-20k polypeptide or functional variant or fragment thereof comprises an amino acid sequence selected from any one of SEQ ID NO: 1 or 3. In another aspect, the GJA1-20k polypeptide or functional variant or fragment thereof further comprises a polyaspartate (D10) peptide tag (SEQ ID NO: 2). In another aspect, the polynucleotide sequence has at least 90-99% identity to SEQ ID NO: 4. In another aspect, the polynucleotide sequence is SEQ ID NO: 4. In another aspect, the GJA1-20k gene expression vector is selected from a viral vector, an adeno-associated virus (AAV) vector, a recombinant AAV (rAAV) vector, a single-stranded AAV vector, a double-stranded AAV vector, a self-complementary AAV (scAAV) vector, or combinations thereof. In another aspect, the GJA1-20k gene expression vector is an AAV vector of a serotype selected from AAV1, AAV2, AAV3, AAV4, AAV5, AAV6, AAV7, AAV8, AAV9, AAV10, AAV11, AAV12, or a hybrid serotype thereof. In another aspect, the GJA1-20k gene expression vector is an AAV9 vector. In another aspect, the cardiac arrhythmia syndrome is selected from arrhythmogenic cardiomyopathy, ischemic arrhythmia, ventricular tachycardia, premature ventricular contractions, right ventricular dysfunction, left ventricular dysfunction, hypertrophic cardiomyopathy, cardiac electrical and physiological dysfunction, or combinations thereof.

Another embodiment described herein is a pharmaceutical composition comprising a GJA1-20k polypeptide or functional variant or fragment thereof. In one aspect, the GJA1-20k polypeptide or functional variant or fragment thereof comprises an amino acid sequence having at least 90-99% identity to any one of SEQ ID NO: 1 or 3. In another aspect, the GJA1-20k polypeptide or functional variant or fragment thereof comprises an amino acid sequence selected from any one of SEQ ID NO: 1 or 3. In another aspect, the GJA1-20k polypeptide or functional variant or fragment thereof further comprises a polyaspartate (D10) peptide tag (SEQ ID NO: 2).

Another embodiment described herein is a pharmaceutical composition comprising a GJA1-20k gene expression vector comprising a polynucleotide sequence encoding a GJA1-20k polypeptide or functional variant or fragment thereof. In one aspect, the polynucleotide sequence has at least 90-99% identity to SEQ ID NO: 4. In another aspect, the polynucleotide sequence is SEQ ID NO: 4. In another aspect, the GJA1-20k gene expression vector is selected from a viral vector, an adeno-associated virus (AAV) vector, a recombinant AAV (rAAV) vector, a single-stranded AAV vector, a double-stranded AAV vector, a self-complementary AAV (scAAV) vector, or combinations thereof. In another aspect, the GJA1-20k gene expression vector is an AAV vector of a serotype selected from AAV1, AAV2, AAV3, AAV4, AAV5, AAV6, AAV7, AAV8, AAV9, AAV10, AAV11, AAV12, or a hybrid serotype thereof. In another aspect, the GJA1-20k gene expression vector is an AAV9 vector.

Another embodiment described herein is the use of a GJA1-20k peptide therapy in a medicament for treating, preventing, reducing the likelihood of having, reducing the severity of, and/or slowing the progression of a cardiac arrhythmia syndrome in a subject. In one aspect, the GJA1-20k peptide therapy comprises a therapeutically effective amount of a pharmaceutical composition comprising: a GJA1-20k polypeptide or functional variant or fragment thereof.

Another embodiment described herein is the use of a GJA1-20k gene therapy in a medicament for treating, preventing, reducing the likelihood of having, reducing the severity of, and/or slowing the progression of a cardiac arrhythmia syndrome in a subject. In one aspect, the GJA1-20k gene therapy comprises a therapeutically effective amount of a pharmaceutical composition comprising: a GJA1-20k gene expression vector comprising a polynucleotide sequence encoding a GJA1-20k polypeptide or functional variant or fragment thereof.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-C show immunoblot protein analysis of human patient heart lysates from patients with nonfailing hearts (NF) and patients with failing arrhythmogenic hearts (ACM). FIG. 1A shows a representative immunoblot of total Cx43, GJA1-20k, and control GAPDH levels in an NF heart lysate and an ACM heart lysate. FIG. 1B shows quantification of the immunoblot protein analysis for GAPDH-normalized total Cx43 levels. FIG. 1C shows quantification of the immunoblot protein analysis for GAPDH-normalized GJA1-20k levels. The data indicate that GJA1-20k protein levels are significantly decreased in the ACM heart lysates compared to the NF heart lysates, while total Cx43 protein levels are not significantly decreased in the ACM patients.

FIG. 2A-C show immunoblot protein analysis of mice heart lysates from wildtype (WT) mice and age/litter-matched Dsg2 mutant mice (DSG). FIG. 2A shows a representative immunoblot of total Cx43, GJA1-20k, and control GAPDH levels in a WT mouse lysate and a DSG mouse lysate. FIG. 2B shows quantification of the immunoblot protein analysis for GAPDH-normalized total Cx43 levels. FIG. 2C shows quantification of the immunoblot protein analysis for GAPDH-normalized GJA1-20k levels. The data indicate that GJA1-20k protein levels are significantly decreased in the DSG mice heart lysates compared to the WT mice heart lysates, while total Cx43 protein levels are not significantly decreased in the DSG mice.

FIG. 3 shows an experimental timeline and protocol schematic for the GJA1-20k gene therapy studies in Dsg2 mutant mice. The WT and DSG mice underwent echocardiography and AAV9 viral injection at 4 weeks of age, followed by telemetry implantation at 18 weeks of age. The mice were sacrificed at 20 weeks of age for histological and biochemical analysis.

FIG. 4A-B show quantification of B mode ejection fraction at the time of AAV9 viral injection (Time 0; 4 weeks of age; FIG. 4A) and at 16 weeks post injection (Time 16 weeks; FIG. 4B). The data indicate that at the time of injection, Dsg2 mice had no detectable change in B mode ejection fraction compared to WT mice. However, at 16 weeks post injection, Dsg2 mice had significantly reduced ejection fraction that was not rescued by GJA1-20k gene therapy.

FIG. 5A-B show the tissue fibrosis effects of GJA1-20k gene therapy in the DSG2 mutant mouse model of ACM. Heart sections were stained with Masson's trichrome to quantify the degree of fibrosis. FIG. 5A shows representative heart images of fibrosis analysis for wildtype (WT) mice treated with GFP alone, DSG2 mutant mice treated with GFP alone, and DSG2 mutant mice treated with GJA1-20k. The bottom images are zoomed-in images of the boxed areas of the top images (scale bars are 10 μm). FIG. 5B shows quantification of the fibrosis image analysis (shown as % fibrosis). The data indicate that fibrosis was significantly increased for DSG2-GFP mice but not for DSG2-GJA1-20k mice, relative to WT control mice.

FIG. 6A-B show the Cx43 molecular effects of GJA1-20k gene therapy in the DSG2 mutant mouse model of ACM using high resolution confocal microscopy. FIG. 6A shows representative immunofluorescence heart micrographs of Cx43 (green) and N-Cadherin (red) expression and localization for wildtype (WT) mice treated with GFP alone (N=6 mice), DSG2 mutant mice treated with GFP alone (N=10 mice), and DSG2 mutant mice treated with GJA1-20k (N=4 mice). Individual cardiomyocytes are outlined in the top images. The bottom images are zoomed-in images of the boxed areas of the top images (scale bars are 10 μm), showing representative intercalated disc (ID) regions. FIG. 6B shows quantification of the immunofluorescence analysis (shown as % ID occupied by Cx43 ). The data were measured as a % of N-Cadherin/Cx43 co-labeling, and 5-10 IDs were measured per mouse. The data indicate that DSG2-GFP mice have significantly less Cx43 at the IDs compared to control WT mice, but GJA1-20k gene therapy recovers Cx43 localization to the IDs in the DSG2 mutant mice.

FIG. 7A-C show the electrophysiological effects of GJA1-20k gene therapy in the DSG2 mutant mouse model of ACM. Nocturnal telemetry recording was conducted, with representative telemetry strips shown in FIG. 7A. FIG. 7A shows representative electrocardiograms (ECGs) for wildtype (WT) mice treated with GFP alone, DSG2 mutant mice treated with GFP alone, and DSG2 mutant mice treated with GJA1-20k. FIG. 7B shows quantification of the telemetry as the number of premature ventricular contractions (PVC)/hour. FIG. 7C shows quantification of the telemetry as a Log transformed arrhythmia score. The data indicate that GJA1-20k gene therapy significantly decreases ventricular ectopy in DSG2 mutant mice as quantified by both PVCs and an arrhythmia score.

FIG. 8A-B show four representative immunohistochemistry (IHC) heart images from an untreated pig (FIG. 8A), and from a pig that was intravenously (IV) administered GJA1-20k peptide at a dose of 0.01 mg/kg for 1 hour (FIG. 8B). Cx43 expression is shown as red stain in both FIG. 8A and 8B. All IHC images are at 100× resolution.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “gene,” “nucleic acid,” “nucleotide,” “polynucleotide,” “oligonucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein. Nucleic acids may be single stranded or double stranded or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

As used herein, “variants” can include, but are not limited to, those that include conservative amino acid (AA) substitution, SNP variants, degenerate variants, and biologically active portions of a gene. A “degenerate variant” as used herein refers to a variant that has a mutated nucleotide sequence, but still encodes the same polypeptide due to the redundancy of the genetic code. There are 20 naturally occurring amino acids; however, some of these share similar characteristics. For example, leucine and isoleucine are both aliphatic, branched, and hydrophobic. Similarly, aspartic acid and glutamic acid are both small and negatively charged. Conservative substitutions in proteins often have a smaller effect on function than non-conservative mutations. Although there are many ways to classify amino acids, they are often sorted into six main groups on the basis of their structure and the general chemical characteristics of their R groups. A mutation among the same class of amino acids is considered a conservative amino acid substitution.

The term “functional” when used in conjunction with “variant” or “fragment” refers to an entity or molecule which possess a biological activity that is substantially similar to a biological activity of the entity or molecule of which it is a variant or fragment thereof. In accordance with the present invention, a GJA1-20k polypeptide may be modified, for example, to facilitate or improve identification, expression, isolation, storage and/or administration, so long as such modifications do not reduce its function to an unacceptable level. In various embodiments, a GJA1-20k polypeptide functional variant or fragment thereof has at least 10%, 20%, 30%, 40%, 50%, 60%, 70%, 80%, 90%, 95%, or 99% of the function of a full-length wildtype GJA1-20k isoform.

As used herein, “substantial identity” of polynucleotide sequences means that a polynucleotide comprises a sequence that has at least 25% sequence identity compared to a reference sequence as determined using programs known in the art (e.g., Basic Local Alignment Search Tool (BLAST)). In preferred embodiments, percent identity can be any integer from 25% to 100%. More preferred embodiments include polynucleotide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. These values can be appropriately adjusted to determine corresponding identity of proteins encoded by two nucleotide sequences by taking into account codon degeneracy, amino acid similarity, reading frame positioning, and the like. Accordingly, polynucleotides of the present invention encoding a protein or polypeptide of the present invention include nucleic acid sequences that have substantial identity to the nucleic acid sequences that encode the proteins or polypeptides of the present invention. Polynucleotides encoding a polypeptide comprising an amino acid sequence that has at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference polypeptide sequence are also preferred.

As used herein, “substantial identity” of amino acid sequences (and of polypeptides having these amino acid sequences) means that an amino acid sequence comprises a sequence that has at least 25% sequence identity compared to a reference sequence as determined using programs known in the art (e.g., BLAST). In preferred embodiments, percent identity can be any integer from 25% to 100%. More preferred embodiments include amino acid or polypeptide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. Polypeptides that are “substantially identical” share amino acid sequences except that residue positions which are not identical may differ by one or more conservative amino acid changes, as described above. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acid substitution groups include valine-leucine-isoleucine, phenylalanine-tyrosine, lysine-arginine, alanine-valine, aspartic acid-glutamic acid, and asparagine-glutamine. Accordingly, polypeptides or proteins, encoded by the polynucleotides of the present invention, include amino acid sequences that have substantial identity to the amino acid sequences of the reference polypeptide sequences.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, therapeutic, often beneficial, effect. In some embodiments, disclosed compositions may further comprise one or more pharmaceutically acceptable carriers or excipients. Example carriers may include, but are not limited to, liposomes, polymeric micelles, microspheres, microparticles, dendrimers, or nanoparticles.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the term “administering” refers to the placement of an agent or a composition as disclosed herein into a subject by a method or route which results in at least partial localization of the agents or composition at a desired site. “Route of administration” may refer to any administration pathway known in the art, including but not limited to oral, intravenous (IV), topical, aerosol, nasal, via inhalation, anal, intra-anal, peri-anal, transmucosal, transdermal, parenteral, enteral, or local. “Parenteral” refers to a route of administration that is generally associated with injection, including intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous (IV), intraarterial, subarachnoid, subcapsular, subcutaneous, transmucosal, or transtracheal. Via the parenteral route, the agent or composition may be in the form of solutions or suspensions for IV infusion or IV injection, or as lyophilized powders. Via the enteral route, the agent or composition can be in the form of capsules, gel capsules, tablets, sugar-coated tablets, syrups, suspensions, solutions, powders, granules, emulsions, microspheres or nanospheres or lipid vesicles or polymer vesicles allowing controlled release. Via the topical route, the agent or composition can be in the form of aerosol, lotion, cream, gel, ointment, suspensions, solutions or emulsions. In one embodiment, the agent or composition may be provided in a powder form and mixed with a liquid, such as water, to form a beverage. In accordance with the present invention, “administering” can be self-administering. For example, it is considered “administering” when a subject consumes a composition as disclosed herein.

As used herein, “contacting” refers to contacting a target cell with an agent (e.g., a GJA1-20k polypeptide or gene expression vector) using any method that is suitable for placing the agent on, in, or adjacent to a target cell. For example, when the cells are in vitro, contacting the cells with the agent can comprise adding the agent to culture medium containing the cells. For example, when the cells are in vivo, contacting the cells with the agent can comprise administering the agent to a subject.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments. In some embodiments of the present invention, a subject is in need of treatment if the subject is suffering from, or at risk of suffering from, one or more cardiac arrhythmia syndromes.

As used herein, a “cardiac arrhythmia syndrome” comprises arrhythmogenic cardiomyopathy (ACM), arrhythmogenic right ventricular cardiomyopathy (ARVC), ischemic arrhythmia, ventricular tachycardia, premature ventricular contractions (PVCs), right ventricular dysfunction, left ventricular dysfunction, hypertrophic cardiomyopathy, cardiac electrical and physiological dysfunction, or combinations thereof. In some embodiments of the present invention, a cardiac arrhythmia syndrome includes both acute and chronic arrhythmic conditions, syndromes, diseases, and disorders.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic manner. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest. In one embodiment of the present invention, a method of treating, preventing, reducing the likelihood of having, reducing the severity of, and/or slowing the progression of a cardiac arrhythmia syndrome in a subject is described.

As used herein, “sample” or “target sample” refers to any sample in which the presence and/or level of a target analyte or target biomarker is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological or bodily fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

As used herein, “target analyte” or “target biomarker” refers to a substance that is associated with a biological state or a biological process, such as a disease state or a diagnostic or prognostic indicator of a disease or disorder (e.g., an indicator identifying the likelihood of the existence or later development of a disease or disorder). The presence or absence of a biomarker, or the increase or decrease in the concentration of a biomarker, can be associated with and/or be indicative of a particular state or process. Biomarkers can include, but are not limited to, cells or cellular components (e.g., a viral cell, a bacterial cell, a fungal cell, a cancer cell, etc.), small molecules, lipids, carbohydrates, nucleic acids, peptides, proteins, enzymes, antigens, and antibodies. A biomarker can be derived from an infectious agent, such as a bacterium, fungus or virus, or can be an endogenous molecule that is found in greater or lesser abundance in a subject suffering from a disease or disorder as compared to a healthy individual (e.g., an increase or decrease in expression of a gene or gene product).

Precise Connexin 43 (Cx43 ) gap junction formation and localization are critical for intercellular communication in the heart as these Cx43 gap junctions provide intercellular coupling, which ensures rapid action potential propagation and synchronized heart contraction. Alterations in Cx43 localization and reductions in gap junction coupling can lead to heart disease and sudden cardiac death due to dangerous ventricular arrhythmias. In fact, most cardiac arrhythmia syndromes trace their cellular origins to a lack of cell-cell Cx43 gap junction coupling.

The GJA1 gene encodes the full-length Cx43 protein (i.e., the longest isoform of GJA1-43k). It has previously been reported that N-terminally truncated Cx43 isoforms exist endogenously in the heart, and that they are produced by alternative translation of the GJA1 mRNA to generate various shorter isoforms including GJA1-32k, GJA1-29k, GJA1-26k, GJA1-20k, GJA1-11k, and GJA1-7k. The most abundant internally translated Cx43 isoform is GJA1-20k, which is an auxiliary subunit for the trafficking of Cx43 in heterologous expression systems and was found to be required for Cx43 trafficking and gap junction formation.

Previous studies have demonstrated that mutating the GJA1 gene to prevent GJA1-20k expression in a CRISPR knockout mouse model is fatal by 4 weeks of age. During electrophysiology monitoring, the mice lacking GJA1-20k expression demonstrated severe abnormal electrocardiograms and frequent arrhythmias despite preserved contractile function, and also exhibited reduced total Cx43 levels and reduced gap junctions. Without GJA1-20k, poorly trafficked Cx43 was degraded. Therefore, these studies suggested that GJA1-20k may provide a valuable therapeutic option for treating certain cardiac arrhythmias in mammals. However, in these previous studies, GJA1-20k gene therapy approaches failed to rescue the knockout mice from sudden cardiac death or improve any of the cardiac electrical abnormalities.

Various embodiments of the present invention provide a pharmaceutical composition comprising a GJA1-20k peptide therapy or a GJA1-20k gene therapy for treating a cardiac arrhythmia syndrome in a subject. In various embodiments, the GJA1-20k peptide or gene is of a mammal. In various embodiments, the GJA1-20k peptide or gene is of a primate, for example, a human, a chimpanzee, a gorilla, or a monkey. In various embodiments, the GJA1-20k peptide or gene is of a horse, a goat, a donkey, a cow, a bull, or a pig. In various embodiments, the GJA1-20k peptide or gene is of a rodent, for example, a mouse, a rat, or a guinea pig. In various embodiments, the GJA1-20k peptide or gene is of a chicken, a duck, a frog, a dog, a cat, or a rabbit.

### Compositions

The present disclosure provides GJA1-20k peptide therapy pharmaceutical compositions comprising a GJA1-20k polypeptide or functional variant or fragment thereof. The present disclosure also provides pharmaceutical compositions comprising a GJA1-20k gene expression vector comprising a polynucleotide sequence encoding a GJA1-20k polypeptide or functional variant or fragment thereof. The disclosed compositions may further comprise one or more pharmaceutically acceptable carriers or excipients.

### GJA1-20k Polypeptide

The disclosed pharmaceutical compositions may comprise a GJA1-20k polypeptide or functional variant or fragment thereof. As used herein, the term “GJA1-20k polypeptide or functional variant or fragment thereof” refers to any polypeptide having an amino acid sequence translated from the C-terminal portion of the GJA1 gene, or a functional variant or fragment of that amino acid sequence.

The GJA1-20k polypeptide may be synthesized and purified using techniques and methods known in the art. For example, the GJA1-20k polypeptide may be synthesized in yeast (e.g., Pichia pastoris) or other suitable host organisms known in the art. In some embodiments, the GJA1-20k polypeptide or functional variant or fragment thereof comprises the human full-length wildtype (WT) GJA1-20k polypeptide sequence (amino acids are numbered according to the full-length human Cx43; that is, the 170 amino acid human full-length GJA1-20k sequence is amino acids 213-382 of human Cx43; SEQ ID NO: 1):

| SEQ ID NO: 1 | | |
|---|---|---|
| Human Full-length Wildtype GJA1-20k Amino Acid Sequence | | |
| 213 | MLVVSLVSLALNIIELFYVFFKGVKDRVKGKSDPYHATSGALSPAKDCGSQKY | 265 |
| 266 | AYFNGCSSPTAPLSPMSPPGYKLVTGDRNNSSCRNYNKQASEQNWANYSA | 315 |
| 316 | EQNRMGQAGSTISNSHAQPEDFPDDNQNSKKLAAGHELQPLAIVDQRPSSR | 366 |
| 367 | ASSRASSRPRPDDLEI | 382 |

In various embodiments, the GJA1-20k polypeptide can be modified for better production, storage, administration, detection, delivery efficiency, etc. For example, in one embodiment, the GJA1-20k polypeptide can be modified with one or more molecular tags and/or linkers. As another example, the GJA1-20k polypeptide can be codon optimized for expression in bacteria and/or yeast. As another example, the GJA1-20k polypeptide can be PEGylated for better stability, better solubility, reduced antigenicity, reduced renal clearance, and prolonged circulatory time. As another example, the GJA1-20k polypeptide can be modified with one or more cell penetrating peptides (CPP). As yet another example, the GJA1-20k polypeptide can be modified with one or more polyaspartate (D10) peptide tags, which aid in biochemical handling (SEQ ID NO: 2):

SEQ ID NO: 2
Polyaspartate (D10) Peptide Tag Sequence
DDDDDDDDDD

In various embodiments, the GJA1-20k polypeptide may comprise one or more mutations from the full-length wildtype GJA1-20k form. For example, in various embodiments, the GJA1-20k polypeptide functional variants are variants with one or more conservative amino acid substitutions, where the variant retains a substantial amount of biological activity. In other embodiments, the GJA1-20k polypeptide may be truncated from the full-length wildtype GJA1-20k form. For example, in one nonlimiting embodiment, the GJA1-20k polypeptide is truncated to remove an N-terminal transmembrane region, where the polypeptide comprises amino acids 236-382 of full-length human Cx43 (GJA1-20k-dTM). In some embodiments, this N-terminal truncation may be critical for GJA1-20k polypeptide synthesis. In one exemplary embodiment, the GJA1-20k polypeptide comprises 180 amino acids comprising an N-terminal poly-His tag, a linker, the truncated GJA1-20k-dTM fragment, and a C-terminal D10 peptide tag (SEQ ID NO: 3):

SEQ ID NO: 3
Example GJA1-20k Polypeptide Sequence
MHHHHHHGGGGSGGGGSVKDRVKGKSDPYHATSGALSPAKDCGSQKYAYF
NGCSSPTAPLSPMSPPGYKLVTGDRNNSSCRNYNKQASEQNWANYSAEQN
RMGQAGSTISNSHAQPFDFPDDNQNSKKLAAGHELQPLAIVDQRPSSRAS
SRASSRPRPDDLEIGGSGGSDDDDDDDDDD

### GJA1-20k Polynucleotide and Gene Expression Vector

The disclosed pharmaceutical compositions may comprise a GJA1-20k gene expression vector comprising a polynucleotide sequence encoding a GJA1-20k polypeptide or functional variant or fragment thereof.

In some embodiments, various gene expression vectors as described herein are used to produce various GJA1-20k polypeptides or functional variants or fragments thereof. For example, various gene expression vectors are introduced into bacteria or yeast to produce various GJA1-20k polypeptides or functional variants thereof, which are later isolated. In various embodiments, the gene expression vector is a plasmid. In various embodiments, the gene expression vector is a viral vector, adeno-associated virus (AAV) vector, recombinant AAV (rAAV) vector, single-stranded AAV vector, double-stranded AAV vector, self-complementary AAV (scAAV) vector, or a combination thereof. In various embodiments, the gene expression vector is a polynucleotide or a virus particle. In various embodiments, the serotype of the virus particle is AAV1, AAV2, AAV3, AAV4, AAV5, AAV6, AAV7, AAV8, AAV9, AAV10, AAV11, AAV12, or a hybrid serotype thereof. In one embodiment, the GJA1-20k gene expression vector is an AAV9 vector containing a CMV promoter, wherein a polynucleotide sequence encoding a GJA1-20k polypeptide or functional variant or fragment thereof is cloned into the AAV9 vector.

In various embodiments, the GJA1-20k gene expression vector comprising a polynucleotide sequence encoding a GJA1-20k polypeptide or functional variant or fragment thereof can be modified for better expression, production, storage, administration, detection, delivery efficiency, etc. In various embodiments, the GJA1-20k gene expression vector may comprise one or more molecular tags and/or linkers. In one embodiment, the GJA1-20k gene expression vector comprises polynucleotide sequences encoding an N-terminal V5 epitope tag and a C-terminal linker. In another embodiment, the GJA1-20k gene expression vector comprises a polynucleotide sequence encoding a C-terminal GFP tag.

In various embodiments, the polynucleotide sequence encoding a GJA1-20k polypeptide or functional variant or fragment thereof may comprise one or more mutations. For example, in various embodiments, the GJA1-20k polynucleotide sequence encodes GJA1-20k polypeptide functional variants having one or more conservative amino acid substitutions, where the variant retains a substantial amount of biological activity. In another example, the polynucleotide sequence includes one or more mutations to prevent the expression of one or more GJA1 isoforms other than GJA1-20k, such as GJA1-11k and/or GJA1-7k. In one aspect, the GJA1-20k polynucleotide sequence includes one or more point mutations to generate a GJA1-20k polypeptide having a M281L mutation (with respect to SEQ ID NO: 1). This M281L mutation specifically prevents the translation of the shorter GJA1-11k and GJA1-7k isoforms. In one exemplary embodiment, the GJA1-20k polynucleotide sequence comprises 534 nucleotides encoding an N-terminal poly-His tag and the full-length GJA1-20k polypeptide sequence having the M281L mutation (SEQ ID NO: 4):

SEQ ID NO: 4
Example GJA1-20k Polynucleotide Sequence
atgcatcatcaccatcaccacatgctggtggtgtccttggtgtccctggc
cttgaatatcattgaactcttctatgttttcttcaagggcgttaaggatc
gggttaagggaaagagcgacccttaccatgcgaccagtggtgcgctgagc
cctgccaaagactgtgggtctcaaaaatatgcttatttcaatggctgctc
ctcaccaaccgctcccctctcgcctctatctcctcctgggtacaagctgg
ttactggcgacagaaacaattcttcttgccgcaattacaacaagcaagca
agtgagcaaaactgggctaattacagtgcagaacaaaatcgactagggca
ggcgggaagcaccatctctaactcccatgcacagccttttgatttccccg
atgataaccagaattctaaaaaactagctgctggacatgaattacagcca
ctagccattgtggaccagcgaccttcaagcagagccagcagtcgtgccag
cagcagacctcggcctgatgacctggagatctag

### Methods of Treatment

The present disclosure also provides a method of treating, preventing, reducing the likelihood of having, reducing the severity of, and/or slowing the progression of a cardiac arrhythmia syndrome in a subject. The method comprises: administering to the subject a therapeutically effective amount of a pharmaceutical composition comprising: a GJA1-20k polypeptide or functional variant or fragment thereof, as described herein; or a GJA1-20k gene expression vector comprising a polynucleotide sequence encoding a GJA1-20k polypeptide or functional variant or fragment thereof, as described herein.

In various embodiments, the pharmaceutical composition comprising the GJA1-20k polypeptide or functional variant or fragment thereof may be administered as a therapeutic agent (e.g., GJA1-20k peptide therapy) to a subject to treat, prevent, reduce the likelihood of having, reduce the severity of, and/or slow the progression of the cardiac arrhythmia syndrome. In various embodiments, the pharmaceutical composition comprising the GJA1-20k gene expression vector comprising a polynucleotide sequence encoding a GJA1-20k polypeptide or functional variant or fragment thereof may be administered as a therapeutic agent (e.g., GJA1-20k gene therapy) to a subject to treat, prevent, reduce the likelihood of having, reduce the severity of, and/or slow the progression of the cardiac arrhythmia syndrome.

In various embodiments, the subject is a human. In various embodiments, the subject is a mammalian subject including but not limited to human, monkey, ape, dog, cat, cow, horse, goat, pig, rabbit, mouse, and rat.

In various embodiments, the cardiac arrhythmia syndrome comprises arrhythmogenic cardiomyopathy (ACM), arrhythmogenic right ventricular cardiomyopathy (ARVC), ischemic arrhythmia, ventricular tachycardia, premature ventricular contractions (PVCs), right ventricular dysfunction, left ventricular dysfunction, hypertrophic cardiomyopathy, cardiac electrical and physiological dysfunction, or combinations thereof. As described herein, the cardiac arrhythmia syndrome may comprise acute and/or chronic arrhythmic conditions, syndromes, diseases, or disorders in a subject. In some embodiments, the cardiac arrhythmia syndrome may be due to ischemic cardiomyopathy. In other embodiments, the cardiac arrhythmia syndrome may be non-ischemic.

In various embodiments, the pharmaceutical composition comprising a GJA1-20k polypeptide or functional variant or fragment thereof or a GJA1-20k gene expression vector comprising a polynucleotide sequence encoding a GJA1-20k polypeptide or functional variant or fragment thereof is administered to the subject by intravenous (IV) injection. Pharmaceutical compositions as described herein may also be administered using alternative routes, including but not limited to intravascular, intraarterial, intramuscular, subcutaneous, intraperitoneal, aerosol, nasal, via inhalation, oral, transmucosal, transdermal, parenteral, implantable pump or reservoir, continuous infusion, enteral application, topical application, local application, capsules and/or injections.

In some embodiments, the subject is administered a single dose of the disclosed pharmaceutical compositions. In other embodiments, the subject is administered a plurality of doses of the disclosed pharmaceutical compositions over a period of time. For example, in various nonlimiting embodiments, a pharmaceutical composition as described herein may be administered to a subject once a day (SID/QD), twice a day (BID), three times a day (TID), four times a day (QID), or more, so as to administer a therapeutically effective amount of the pharmaceutical composition to the subject, where the therapeutically effective amount is any one or more of the doses described herein. In some embodiments, a pharmaceutical composition as described herein is administered to a subject 1-3 times per day, 1-7 times per week, 1-9 times per month, 1-12 times per year, or more. In other embodiments, a pharmaceutical composition as described herein is administered for about 1-10 days, 10-20 days, 20-30 days, 30-40 days, 40-50 days, 50-60 days, 60-70 days, 70-80 days, 80-90 days, 90-100 days, 1-6 months, 6-12 months, 1-5 years, or more. In various embodiments, a pharmaceutical composition as described herein is administered at about 0.001-0.01, 0.01-0.1, 0.1-0.5, 0.5-5, 5-10, 10-20, 20-50, 50-100, 100 -200, 200-300, 300-400, 400-500, 500-600, 600-700, 700-800, 800-900, 900-1000 mg/kg, or a combination thereof.

The actual dosing regimen can depend upon many factors, including but not limited to the judgment of a trained physician, the overall condition of the subject, and the specific type of cardiac arrhythmia syndrome. The actual dosage can also depend on the determined experimental effectiveness of the specific pharmaceutical composition (e.g., a GJA1-20k polypeptide or functional variant or fragment thereof; a GJA1-20k gene expression vector comprising a polynucleotide sequence encoding a GJA1-20k polypeptide or functional variant or fragment thereof) that is administered. For example, the dosage may be determined based on in vitro responsiveness of relevant cultured cells, or in vivo responses observed in appropriate animal models or human studies for both the GJA1-20k peptide therapy and the GJA1-20k gene therapy.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:

Clause 1. A method of treating, preventing, reducing the likelihood of having, reducing the severity of, and/or slowing the progression of a cardiac arrhythmia syndrome in a subject, the method comprising:
administering to the subject a therapeutically effective amount of a pharmaceutical composition comprising:
   a GJA1-20k polypeptide or functional variant or fragment thereof; or
   a GJA1-20k gene expression vector comprising a polynucleotide sequence encoding a GJA1-20k polypeptide or functional variant or fragment thereof.

Clause 2. The method of clause 1, wherein the pharmaceutical composition is administered to the subject by intravenous (IV) injection.

Clause 3. The method of clause 1 or 2, wherein the GJA1-20k polypeptide or functional variant or fragment thereof comprises an amino acid sequence having at least 90-99% identity to any one of SEQ ID NO: 1 or 3.

Clause 4. The method of any one of clauses 1-3, wherein the GJA1-20k polypeptide or functional variant or fragment thereof comprises an amino acid sequence selected from any one of SEQ ID NO: 1 or 3.

Clause 5. The method of any one of clauses 1-4, wherein the GJA1-20k polypeptide or functional variant or fragment thereof further comprises a polyaspartate (D10) peptide tag (SEQ ID NO: 2).

Clause 6. The method of any one of clauses 1-5, wherein the polynucleotide sequence has at least 90-99% identity to SEQ ID NO: 4.

Clause 7. The method of any one of clauses 1-6, wherein the polynucleotide sequence is SEQ ID NO: 4.

Clause 8. The method of any one of clauses 1-7, wherein the GJA1-20k gene expression vector is selected from a viral vector, an adeno-associated virus (AAV) vector, a recombinant AAV (rAAV) vector, a single-stranded AAV vector, a double-stranded AAV vector, a self-complementary AAV (scAAV) vector, or combinations thereof.

Clause 9. The method of any one of clauses 1-8, wherein the GJA1-20k gene expression vector is an AAV vector of a serotype selected from AAV1, AAV2, AAV3, AAV4, AAV5, AAV6, AAV7, AAV8, AAV9, AAV10, AAV11, AAV12, or a hybrid serotype thereof.

Clause 10. The method of any one of clauses 1-9, wherein the GJA1-20k gene expression vector is an AAV9 vector.

Clause 11. The method of any one of clauses 1-10, wherein the cardiac arrhythmia syndrome is selected from arrhythmogenic cardiomyopathy, ischemic arrhythmia, ventricular tachycardia, premature ventricular contractions, right ventricular dysfunction, left ventricular dysfunction, hypertrophic cardiomyopathy, cardiac electrical and physiological dysfunction, or combinations thereof.

Clause 12. A pharmaceutical composition comprising a GJA1-20k polypeptide or functional variant or fragment thereof.

Clause 13. The pharmaceutical composition of clause 12, wherein the GJA1-20k polypeptide or functional variant or fragment thereof comprises an amino acid sequence having at least 90-99% identity to any one of SEQ ID NO: 1 or 3.

Clause 14. The pharmaceutical composition of clause 12 or 13, wherein the GJA1-20k polypeptide or functional variant or fragment thereof comprises an amino acid sequence selected from any one of SEQ ID NO: 1 or 3.

Clause 15. The pharmaceutical composition of any one of clauses 12-14, wherein the GJA1-20k polypeptide or functional variant or fragment thereof further comprises a polyaspartate (D10) peptide tag (SEQ ID NO: 2).

Clause 16. A pharmaceutical composition comprising a GJA1-20k gene expression vector comprising a polynucleotide sequence encoding a GJA1-20k polypeptide or functional variant or fragment thereof.

Clause 17. The pharmaceutical composition of clause 16, wherein the polynucleotide sequence has at least 90-99% identity to SEQ ID NO: 4.

Clause 18. The pharmaceutical composition of clause 16 or 17, wherein the polynucleotide sequence is SEQ ID NO: 4.

Clause 19. The pharmaceutical composition of any one of clauses 16-18, wherein the GJA1-20k gene expression vector is selected from a viral vector, an adeno-associated virus (AAV) vector, a recombinant AAV (rAAV) vector, a single-stranded AAV vector, a double-stranded AAV vector, a self-complementary AAV (scAAV) vector, or combinations thereof.

Clause 20. The pharmaceutical composition of any one of clauses 16-19, wherein the GJA1-20k gene expression vector is an AAV vector of a serotype selected from AAV1, AAV2, AAV3, AAV4, AAV5, AAV6, AAV7, AAV8, AAV9, AAV10, AAV11, AAV12, or a hybrid serotype thereof.

Clause 21. The pharmaceutical composition of any one of clauses 16-20, wherein the GJA1-20k gene expression vector is an AAV9 vector.

Clause 22. Use of a GJA1-20k peptide therapy in a medicament for treating, preventing, reducing the likelihood of having, reducing the severity of, and/or slowing the progression of a cardiac arrhythmia syndrome in a subject.

Clause 23. The use of clause 22, wherein the GJA1-20k peptide therapy comprises a therapeutically effective amount of a pharmaceutical composition comprising:
a GJA1-20k polypeptide or functional variant or fragment thereof.

Clause 24. Use of a GJA1-20k gene therapy in a medicament for treating, preventing, reducing the likelihood of having, reducing the severity of, and/or slowing the progression of a cardiac arrhythmia syndrome in a subject.

Clause 25. The use of clause 24, wherein the GJA1-20k gene therapy comprises a therapeutically effective amount of a pharmaceutical composition comprising:
a GJA1-20k gene expression vector comprising a polynucleotide sequence encoding a GJA1-20k polypeptide or functional variant or fragment thereof.

### EXAMPLES

### Example 1

### GJA1-20k Protein Expression in Arrhythmogenic Cardiomyopathy (ACM) Patient Hearts

Arrhythmogenic cardiomyopathy (ACM) is an under-recognized heritable cause of sudden cardiac death without effective treatment options. The prevalence of ACM is about 1 in every 2,000-5,000 individuals. Furthermore, 11% of all sudden cardiac death cases in young adults and 22% of all cases in athletes are attributed to ACM. While the disease was originally named arrhythmogenic right ventricular dysplasia after autopsy studies revealed fibrofatty replacement of the right ventricle, the nomenclature has since been updated to reflect a genetic disease with structurally normal heart at birth that can perturb myocytes in both ventricles and increase the risk for sudden death. In many other cardiomyopathies such as hypertrophic or dilated cardiomyopathy, the risk of death is related to histological degree of myocyte disarray, amount of scar present, cardiac chamber remodeling, or degree of contractile dysfunction. This is not the case in ACM patients in which malignant ventricular arrhythmias often precede histologic changes in heart muscle or change in ventricular function.

ACM is caused by mutations of junctional proteins resulting in heart rhythm abnormalities, heart failure, and sudden cardiac death. The majority of cases of ACM can be attributed to mutations in desmosome-associated proteins such as desmoglein 2 (Dsg2). How mutations in structural disc components cause arrhythmias prior to visible structural changes in the myocytes themselves is not well established. However, ACM myocytes have been found to exhibit impaired trafficking of the gap junction protein Connexin 43 (Cx43 ) to the intercalated discs of the heart, a characteristic that is associated with sudden death.

It has previously been demonstrated that the Cx43 gene, Gja1, undergoes internal translation to form multiple truncated isoforms from the full-length protein. Additionally, the trafficking of cardiac Cx43 to the intercalated discs was previously identified to be dependent on the truncated 20 kDa isoform of Cx43 (GJA1-20k), which is generated by internal translation initiation of the Gja1 mRNA.

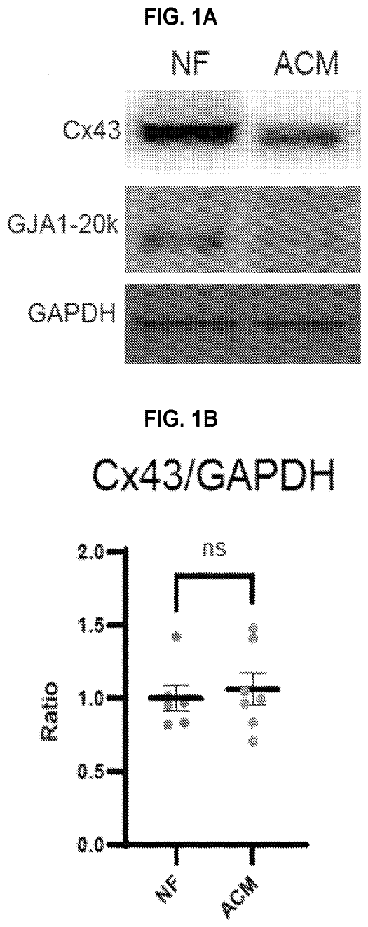

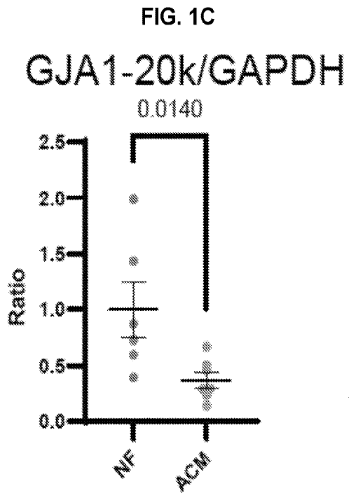

Total Cx43 and GJA1-20k protein levels were investigated in the hearts of patients fulfilling criteria for ACM and who underwent heart transplant. Biochemical evaluation of lysates from the right ventricles of the ACM and donor control hearts indicated no significant difference in total Cx43 levels between the two groups, but a significant reduction in GJA1-20k expression was observed in the ACM hearts (FIG. 1A-C). It was both surprising and unexpected to discover that GJA1-20k was reduced in the peri-transplant ACM hearts because patients and animal models with ischemic cardiomyopathy are known to have increased GJA1-20k protein levels rather than decreased, as GJA1-20k is generally a stress response protein.

### Example 2

### GJA1-20k Gene Therapy in the DSG2 Mutant Mouse Model of ACM

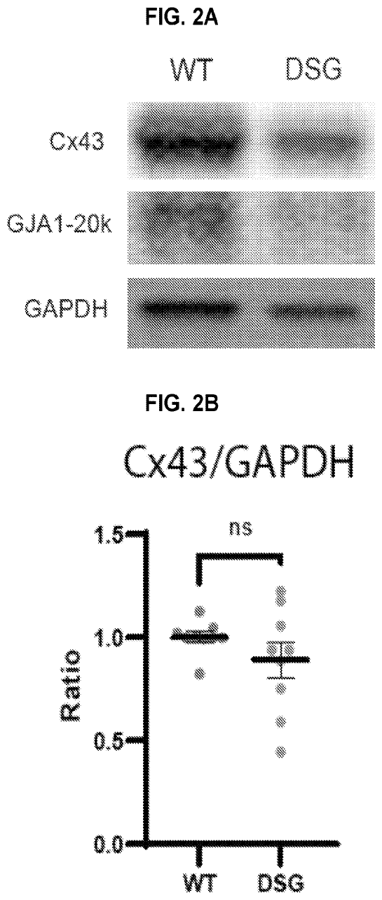

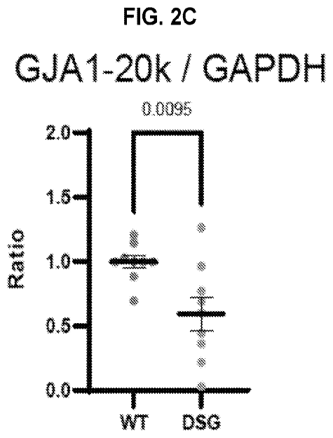

Desmoglein 2 (DSG2) homozygous knock-out mutant mice (DSG2-KO; Dsg2−/−) produce a typical ACM phenotype, making these mice a well-established model for studying ACM. As discussed in Example 1, in general, GJA1-20k is a stress response protein, having increased expression in certain stress states and diseases including heart failure and ischemia. However, similar to the human patient data of FIG. 1A-C, DSG2 mutant mice hearts were also unexpectedly found to have decreased expression levels of GJA1-20k compared to control age/litter-matched WT mice hearts, while changes in total Cx43 levels were insignificant (FIG. 2A-C). It was therefore hypothesized that GJA1-20k gene therapy could reduce dysrhythmias by improving the trafficking of Cx43 to the intercalated disc in the DSG2 mutant mouse model of ACM.

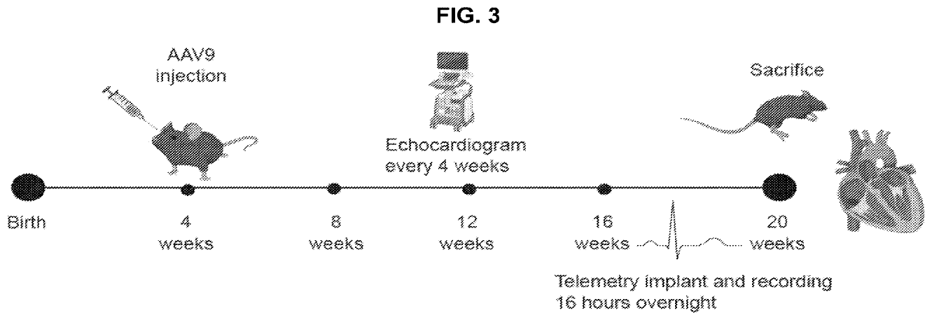

At four weeks of age, the DSG2-KO mutant mice and wildtype (WT) littermate controls were injected with an AAV9 viral expression vector containing either GFP alone or GFP linked to a GJA1-20k polynucleotide sequence (SEQ ID NO: 4). Echocardiography (ECG) was performed every four weeks with telemetry implantation at 18 weeks of age to record cardiac electrical activity over a continuous 16-hour overnight period. At 20 weeks of age, the mice were sacrificed, and hearts were harvested for further analysis, including histological and biochemical analysis. An experimental schematic is shown in FIG. 3. Hearts were subjected to RT-PCR for a GFP segment and samples were excluded from analysis if GFP amplification was not detectable (defined as less than 28 RT-PCR cycles).

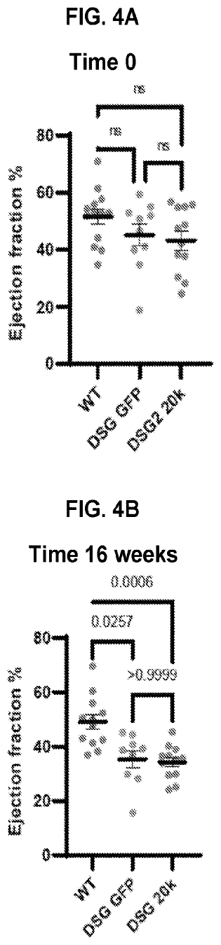

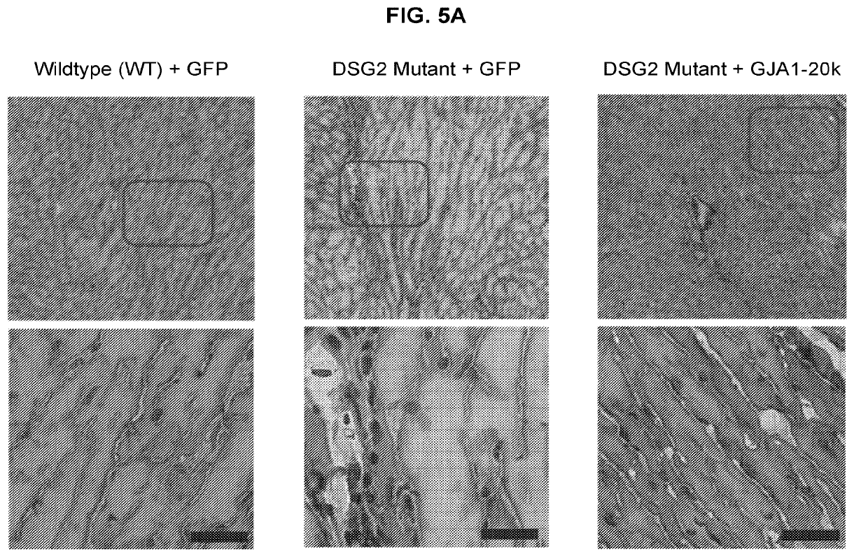

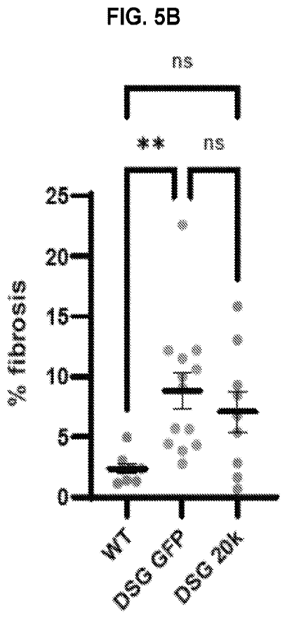

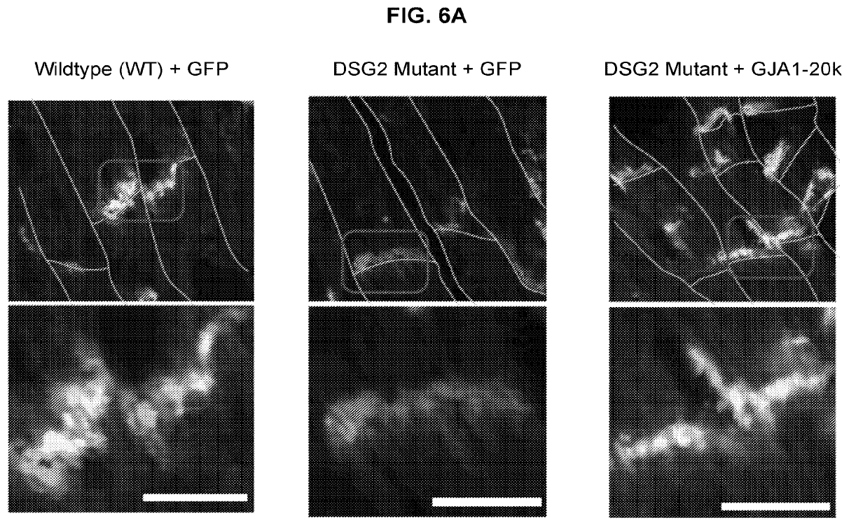

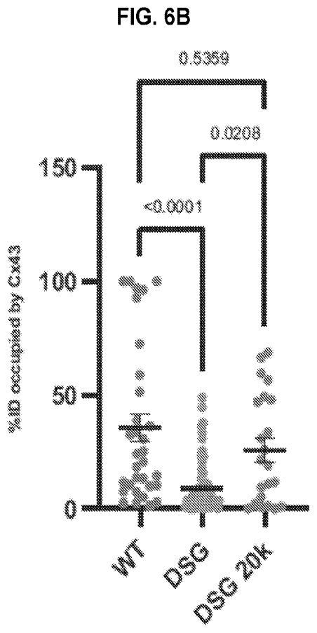

Compared to WT mice, at the time of AAV9 injection, DSG2-KO mice had no detectable change in ejection fraction; however, at 16 weeks post injection, DSG2-KO mice had reduced ejection fraction without rescue by GJA1-20k (FIG. 4A-B). Heart sections were stained with Masson's trichrome to quantify the degree of fibrosis between the different mice. Fibrosis was increased in DSG2-KO mice relative to WT control mice, yet unaffected by GJA1-20k gene therapy (FIG. 5A-B). Quantitative immunofluorescence analysis of the mice hearts using high resolution confocal microscopy revealed an increase in Cx43 localization at the intercalated disc (ID), measured as a % of N-Cadherin/Cx43 co-labeling, in mice treated with GJA1-20k relative to GFP-treated (25% vs. 6%, respectively; N=5-10 ID images/mouse). These data are shown in FIG. 6A-B.

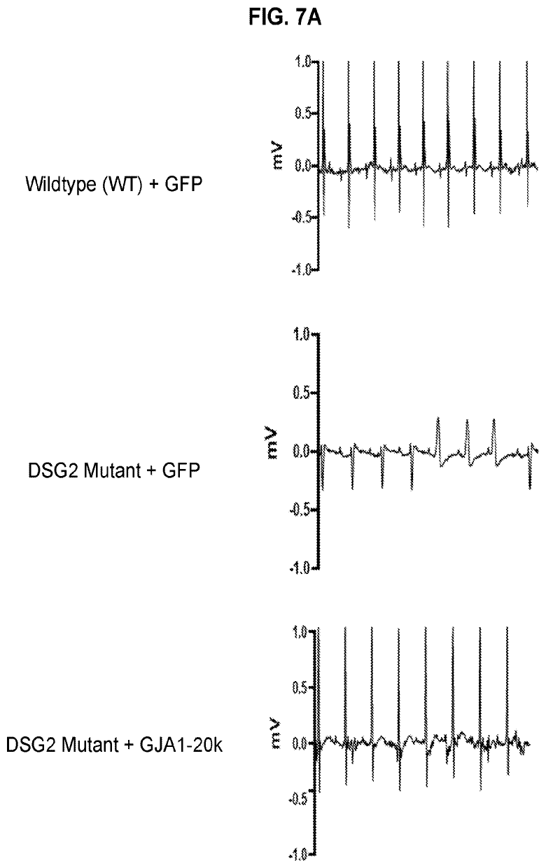

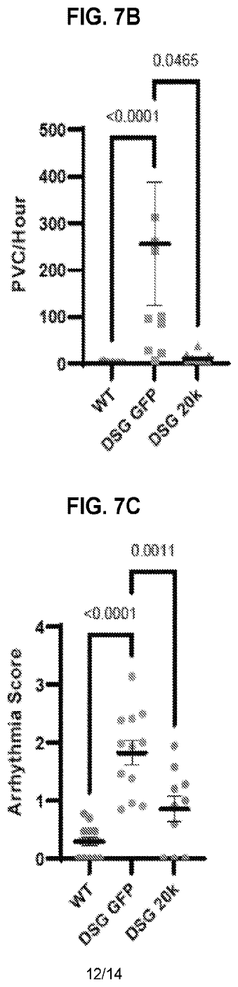

Furthermore, compared to their WT littermates, the DSG2-KO mice that received a GFP viral vector demonstrated a significant increase in the mean number of premature ventricular contractions (PVCs) over a 16-hour period of nocturnal telemetry recording (FIG. 7A-C). In contrast, the DSG2-KO mice that received the GJA1-20k-GFP viral vector demonstrated significantly fewer ventricular arrhythmias as quantified by both PVC/hour and a Log transformed arrhythmia score (FIG. 7A-C).

These results demonstrate that GJA1-20k gene therapy significantly restores Cx43 trafficking to the intercalated disc and rescues ACM hearts from their arrhythmogenic phenotype, independent of left ventricular dysfunction or fibrosis. This study also provides the first identifiable disease state animal model with decreased GJA1-20k expression levels in adulthood. The rescue of Cx43 localization to the disc was found to be associated with a reduction in arrhythmias in the DSG2 mutant mice. Overall, this study demonstrates that the administration of an exogenous GJA1-20k gene therapy may represent a potential therapeutic approach for the prevention of arrhythmogenic right ventricular cardiomyopathy (ARVC)-associated ventricular arrhythmias. Gene therapy of GJA1-20k improves endogenous localization of Cx43 to intercalated discs better than adding exogenous Cx43 because the Cx43 trafficking pathway is restored with GJA1-20k. Future research will explore the duration by which a single application of GJA1-20k gene therapy can protect against ventricular arrhythmias and whether, in humans, GJA1-20k gene therapy should be administered intravenously or directly into myocardium.

### Example 3

### Intravenous GJA1-20k Peptide Therapy in a Pig Model

To investigate whether the disclosed GJA1-20k peptide therapy could produce a molecular and/or therapeutic effect in vivo, Sus scrofa wild boar pigs (Yorkshire mix breed) were IV administered a GJA1-20k polypeptide (SEQ ID NO: 3). The age of the pigs in this study ranged from 4-6 months old, the weights ranged from 50-75 kg, and the study included intact females and castrated males at a 1:1 ratio.

### General Anesthesia

The pigs were anesthetized using a general anesthesia protocol. Animals were fasted for 8-12 hours before induction of anesthesia. The animals were allowed free access to water and were anesthetized according to Table 1, shown below.

TABLE 1
Anesthesia protocol for pig GJA1-20k peptide study

| Agent | Dose | Route | Anesthesia Phase |
|---|---|---|---|
| Telazol | 4.4 mg/kg | IM | Induction |
| Ketamine | 2.2 mg/kg | IM | Induction |
| Xylazine | 2.2 mg/kg | IM | Induction |
| Hydromorphone | 0.1 mg/kg | IN | Induction |
| Isoflurane | 1.5-3.0% | Inhaled | Maintenance |

Endotracheal intubation of the pigs was achieved with a cuffed endotracheal tube. Isoflurane (5% in oxygen at 2 L/min) was administered by mask if injectable anesthesia induction was of insufficient depth to accomplish endotracheal intubation. After intubation, gaseous isoflurane anesthesia was delivered at approximately 1.5-3.0% in 2 L/min of oxygen. Animals were mechanically ventilated with a fraction of inspired oxygen of 30-100% (to maintain a pulse oximetry reading 95-99%), a positive end-expiratory pressure of 4 cmH2O, tidal volumes of 6-8 mL/kg, and a variable respiratory rate to maintain an end-tidal CO₂ of 35-45 mmHg. Balanced isotonic fluids (Plasmalyte 148, Baxter, IL) was administered intravenously at 5 ml/kg/hour. Animals were positioned in dorsal recumbency followed by placement of electrocardiogram leads, as well as temperature and pulse oximetry probes. All animals were kept on warming blankets set to 39° C. to minimize hypothermia. Reflective blankets were used as needed to maintain body temperature. The depth of anesthesia was evaluated by jaw tone, eye position, palpebral reflex approximately every 15 minutes, and absence of spontaneous movement throughout.

### Vascular Access and Surgical Preparation

The skin of the animals was cleaned with three alternating scrubs consisting of chlorhexidine or betadine, and then alcohol. After the third scrub, a final application of chlorhexidine or betadine was applied and allowed to dry completely. After the chlorhexidine or betadine had dried, each surgical site was draped sterilely. All vascular access sites were infiltrated with 2% lidocaine subcutaneously. The following catheters were inserted using ultrasound guidance and a Seldinger technique:

Two 7 Fr femoral arterial lines to 1) sample blood, record blood pressure continuously, and 2) introduce the resuscitative endovascular balloon occlusion of the aorta (REBOA) catheter, respectively;
a 5 Fr carotid arterial line to measure blood pressure above the point of occlusion;
a dual lumen resuscitation line in an internal jugular vein to administer whole blood;
a triple lumen catheter in the contralateral internal jugular vein to monitor central venous pressure; and
a 9 Fr femoral vein catheter to administer crystalloid boluses during the resuscitation phase.

The REBOA catheter (ER-REBOA, Prytime Medical, TX) was inserted to place the balloon immediately superior to the diaphragm, with location confirmed by fluoroscopy. Throughout the experiment, mean arterial pressure (MAP) above and below the balloon and central venous pressure was recorded continuously (PowerLab data acquisition platform, AD Instruments, CO). A midline incision was performed. The spleen was removed using electrocautery, sutures, and/or a surgical stapler. A Foley catheter was inserted into the bladder via a cystotomy and connected to a close collection system. The goal MAP before starting experimental procedures was 65 mmHg. Therefore, after instrumentation, the animal was given up to two 5 ml/kg bolus of Plasmalyte 148 if the MAP was below 65 mmHg. If the MAP remained below 65 mmHg, a continuous intravenous infusion of norepinephrine (Norepinephrine, Hospira, IL) (0.01-0.2 mcg/kg/min) was then initiated.

### Hemorrhagic Shock

Hemorrhagic shock was induced via removal of 25% of the animals estimated blood volume [25%×60 (mL/kg)×body weight (kg); blood losses during experimental setup were quantified and subtracted from the volume of blood to be removed] over 30 minutes through a femoral arterial line. Shed blood was collected in citrated blood collection bags under constant agitation and then stored in a water bath at 37° C. Following the 30-minute hemorrhage phase (T30), animals remained in a hypotensive state for an additional 30 minutes (T60).

### Aortic Occlusion and GJA1-20k Administration

At T60, animals were subjected to 45 minutes of REBOA with GJA1-20k (R+/GJA1+) or without GJA1-20k (R+/GJA−) administration. Complete aortic occlusion was confirmed via loss of pulsatility on the distal arterial waveform, which was continuously monitored. The balloon was deflated over 10 minutes (T105-T115). GJA1-20k peptide therapy (SEQ ID NO: 3) was IV administered at 0.01 mg/kg over a 1-hour period (T85-T145).

In a separate independent dose-finding pilot study, four pigs were IV administered a dose of 0.1 mg/kg. This resulted in a 50% mortality rate. In the two surviving pigs, the volume of isotonic crystalloids required throughout the experiment was smaller than that in the control group.

### Critical Care Period

From T100-T115, animals were transfused with their shed blood. Intravenous calcium (21 mL of calcium gluconate) was administered over 10 minutes to prevent citrate-induced hypocalcemia. At T105, the balloon was deflated over 10 minutes. Animals then entered the critical care phase, where they were resuscitated with a combination of balanced isotonic crystalloids (Plasmalyte 148) and norepinephrine to maintain a MAP >65 mmHg according to a pre-specified algorithm:
If MAP <65 mmHg and:
Central venous pressure is <7 mmHg: intravenous bolus of 5 mL/kg of Plasmalyte 148;
Central venous pressure is 7-11 mmHg: intravenous bolus of 5 mL/kg of Plasmalyte 148 and increase norepinephrine infusion by 0.01 mcg/kg/min; or
Central venous pressure is >11 mmHg: increase norepinephrine infusion by 0.01 mcg/kg/min.

The norepinephrine infusion was titrated down by 0.01 mcg/kg/min increments if the MAP was >75 mmHg. Throughout the experiment, hypoglycemia, hyperkalemia, and ionized hypocalcemia animals were treated as needed. Animals were monitored for a total experimental time of 6 hours after the start of the controlled hemorrhage.

### Tissue Sampling

Arterial blood was sampled at baseline, T0, T30, T60, T105, T120, T180, T240, and T360. A complete blood count (hemoglobin concentration, red blood cell count, white blood cell count with differential, and platelet count) was performed at baseline and at the end of the experiment. At each time point, blood gases (O₂, CO₂) tension, pH, and concentrations of bicarbonate, lactate, glucose, and electrolytes (sodium, potassium, calcium, chloride) was measured using a point-of-care platform (iStat, Abbott Laboratories, IL). Biochemistry panels (BUN, creatinine, aspartate aminotransferase, alanine aminotransferase, alkaline phosphatase) were also measured using the same point-of-care platform at each time point. Blood was centrifuged, the serum isolated, and immediately stored at −80° C. for enzyme-linked immunosorbent assays (ELISAs) and troponin I quantification.

Tissue from the following organs was harvested at the end of the experiment after a routine necropsy: left and right ventricle, renal cortex and medulla, liver, and duodenum.

### Results

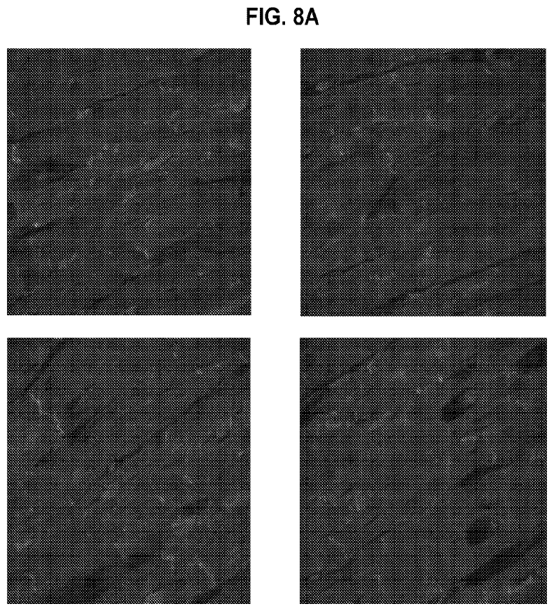

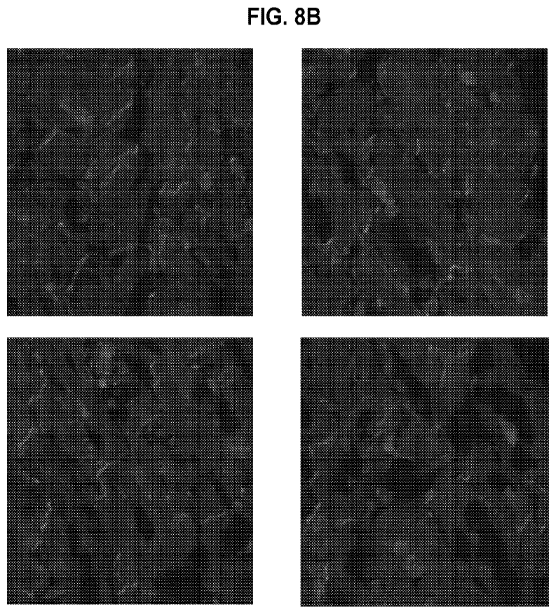

Intravenous administration of the GJA1-20k peptide in pigs was found to rapidly increase Cx43 expression in the intercalated disc of the heart, indicating increased Cx43 gap junction coupling. FIG. 8A-B show representative data of Cx43 gap junctions at the intercalated disc from pig hearts treated with and without the GJA1-20k peptide. In the hearts treated with the GJA1-20k peptide, there is much more Cx43 observed at the intercalated disc (longitudinal ends of cell-cell borders) than there is inside the cell. This indicates better Cx43 cell-cell coupling when GJA1-20k is administered to the pigs.

Successful intravenous GJA1-20k peptide therapy has never been previously reported. Other peptides that have been synthesized to the C-terminal region of Cx43 comprising various different portions of GJA1-20k have failed to show a molecular or therapeutic effect. Therefore, it was surprising to discover that the GJA1-20k peptide was able to enter cells within the blood stream and that peripheral IV injection of the peptide reached the heart in the pigs.

1. A method of treating, preventing, reducing the likelihood of having, reducing the severity of, and/or slowing the progression of a cardiac arrhythmia syndrome in a subject, the method comprising:
administering to the subject a therapeutically effective amount of a pharmaceutical composition comprising:
a GJA1-20k polypeptide or functional variant or fragment thereof; or
a GJA1-20k gene expression vector comprising a polynucleotide sequence encoding a GJA1-20k polypeptide or functional variant or fragment thereof.

2. The method of claim 1, wherein the pharmaceutical composition is administered to the subject by intravenous (IV) injection.

3. The method of claim 1, wherein the GJA1-20k polypeptide or functional variant or fragment thereof comprises an amino acid sequence having at least 90-99% identity to any one of SEQ ID NO: 1 or 3.

4. The method of claim 1, wherein the GJA1-20k polypeptide or functional variant or fragment thereof comprises an amino acid sequence selected from any one of SEQ ID NO: 1 or 3.

5. The method of claim 1, wherein the GJA1-20k polypeptide or functional variant or fragment thereof further comprises a polyaspartate (D10) peptide tag (SEQ ID NO: 2).

6. The method of claim 1, wherein the polynucleotide sequence has at least 90-99% identity to SEQ ID NO: 4.

7. The method of claim 1, wherein the polynucleotide sequence is SEQ ID NO: 4.

8. The method of claim 1, wherein the GJA1-20k gene expression vector is selected from a viral vector, an adeno-associated virus (AAV) vector, a recombinant AAV (rAAV) vector, a single-stranded AAV vector, a double-stranded AAV vector, a self-complementary AAV (scAAV) vector, or combinations thereof.

9. The method of claim 8, wherein the GJA1-20k gene expression vector is an AAV vector of a serotype selected from AAV1, AAV2, AAV3, AAV4, AAV5, AAV6, AAV7, AAV8, AAV9, AAV10, AAV11, AAV12, or a hybrid serotype thereof.

10. The method of claim 9, wherein the GJA1-20k gene expression vector is an AAV9 vector.

11. The method of claim 1, wherein the cardiac arrhythmia syndrome is selected from arrhythmogenic cardiomyopathy, ischemic arrhythmia, ventricular tachycardia, premature ventricular contractions, right ventricular dysfunction, left ventricular dysfunction, hypertrophic cardiomyopathy, cardiac electrical and physiological dysfunction, or combinations thereof.

12. A pharmaceutical composition comprising a GJA1-20k polypeptide or functional variant or fragment thereof.

13. The pharmaceutical composition of claim 12, wherein the GJA1-20k polypeptide or functional variant or fragment thereof comprises an amino acid sequence having at least 90-99% identity to any one of SEQ ID NO: 1 or 3.

14. The pharmaceutical composition of claim 12, wherein the GJA1-20k polypeptide or functional variant or fragment thereof comprises an amino acid sequence selected from any one of SEQ ID NO: 1 or 3.

15. The pharmaceutical composition of claim 12, wherein the GJA1-20k polypeptide or functional variant or fragment thereof further comprises a polyaspartate (D10) peptide tag (SEQ ID NO: 2).

16. A pharmaceutical composition comprising a GJA1-20k gene expression vector comprising a polynucleotide sequence encoding a GJA1-20k polypeptide or functional variant or fragment thereof.

17. The pharmaceutical composition of claim 16, wherein the polynucleotide sequence has at least 90-99% identity to SEQ ID NO: 4.

18. The pharmaceutical composition of claim 16, wherein the polynucleotide sequence is SEQ ID NO: 4.

19. The pharmaceutical composition of claim 16, wherein the GJA1-20k gene expression vector is selected from a viral vector, an adeno-associated virus (AAV) vector, a recombinant AAV (rAAV) vector, a single-stranded AAV vector, a double-stranded AAV vector, a self-complementary AAV (scAAV) vector, or combinations thereof.

20. The pharmaceutical composition of claim 19, wherein the GJA1-20k gene expression vector is an AAV vector of a serotype selected from AAV1, AAV2, AAV3, AAV4, AAV5, AAV6, AAV7, AAV8, AAV9, AAV10, AAV11, AAV12, or a hybrid serotype thereof.

21. The pharmaceutical composition of claim 20, wherein the GJA1-20k gene expression vector is an AAV9 vector.

22-25. (canceled)
